(19) United States
(12) Patent Application Publication
(10) Pub. No.: US 2021/0386789 A1
(43) Pub. Date: Dec. 16, 2021

(54) Enhancement of Mesenchymal Stem Cell Anti-inflammatory and Regenerative Activity Using mTOR Inhibitors

(71) Applicant: BRAIN CANCER RESEARCH INSTITUTE, San Diego, CA (US)

(72) Inventors: Thomas Ichim, San Diego, CA (US); Feng Lin, San Diego, CA (US); Sandeep Pingle, San Diego, CA (US); Shashanka Ashili, Rosemount, MN (US)

(73) Assignee: BRAIN CANCER RESEARCH INSTITUTE, San Diego, CA (US)

(21) Appl. No.: 17/345,885

(22) Filed: Jun. 11, 2021

Related U.S. Application Data
(60) Provisional application No. 63/038,045, filed on Jun. 11, 2020.

(51) Int. Cl. A61K 35/28; A61K 31/436; A61K 31/675; C12N 5/0775

(52) U.S. Cl. CPC A61K 35/28; A61K 31/436; C12N 2501/727; C12N 5/0663; A61K 31/675

(57) ABSTRACT

The invention teaches the unexpected finding that treatment of mesenchymal stem cells with inhibitors of mammalian target of rapamycin (mTOR) lead to enhancement of regenerative and/or anti-inflammatory activity of said stem cells. In one embodiment, rapamycin treatment of mesenchymal stem cells (MSC) is associated with enhanced basal and stimulated production of therapeutic factors. In one embodiment other regenerative activities are enhanced by treatment with inhibitors of mTOR such as angiogenesis, neurogenesis, protection from apoptosis, and immune modulation.

### CROSS-REFERENCE TO RELATED APPLICATIONS

This application claims the benefit or priority to U.S. Provisional Application No. 63/038,045, filed Jun. 11, 2020, the contents of which are incorporated herein by reference in its entirety.

### FIELD OF THE INVENTION

The teachings herein relate to methods of augmenting the regenerative activity of mesenchymal stem cells by contacting and mixing said mesenchymal stem cells with one or more inhibitors of mammalian target of rapamycin.

### BACKGROUND OF THE INVENTION

Stem cell therapy offers the possibility of regenerative medicine, which conceptually can revolutionize the treatment of chronic disease. Mesenchymal stem cells (MSC) are a clinical grade regenerative cell population which has been demonstrated to possess therapeutic effects in a wide variety of inflammatory and degenerative conditions. MSC have been derived from tissue selected from the group consisting of the placenta, cord blood, Wharton's Jelly, menstrual blood, endometrium, skin, omentum, amniotic fluid, adipose tissue, bone marrow, umbilical cord tissue, peripheral blood, hair follicle, and a mixture thereof.

Unfortunately, despite the therapeutic promise of MSC, numerous clinical trials have failed, in part due to lack of sufficient therapeutic efficacy of said cells. In the current disclosure we provide novel means of enhancing MSC activity through the use inhibitors of mTOR such as rapamycin. Rapamycin (sirolimus, RAPA) is a bacterial macrolide that forms a complex with FK-binding protein (FKBP-12) that in turn binds to the mammalian target of rapamycin (mTOR) with high affinity.

### SUMMARY

Preferred methods herein are directed to augmenting regenerative activity of mesenchymal stem cells comprising contacting/mixing said mesenchymal stem cell with one or more inhibitors of mammalian target of rapamycin (mTOR).

Preferred methods include embodiments wherein said mesenchymal stem cells express markers selected from a group comprising of: a) CD90; b) CD105 and c) CD74.

Preferred methods include embodiments wherein said mesenchymal stem cells lack expression of markers selected from a group comprising of: a) CD14; b) CD45 and c) CD34.

Preferred methods include embodiments wherein said mesenchymal stem cells are plastic adherent.

Preferred methods include embodiments wherein said mesenchymal stem cells are selected from a group of tissues comprising of: a) bone marrow b) placenta; c) menstrual blood; d) peripheral blood; e) adipose tissue; f) umbilical cord blood; g) Wharton's jelly; and h) fallopian tube.

Preferred methods include embodiments wherein said peripheral blood is drawn after subject is treated with one or more agents capable of mobilizing bone marrow derived mesenchymal stem cells.

Preferred methods include embodiments wherein said mobilizing agent is G-CSF.

Preferred methods include embodiments wherein said mobilizing agent is GM-CSF.

Preferred methods include embodiments wherein said mobilizing agent is M-CSF.

Preferred methods include embodiments wherein said mobilizing agent is FLT-3 ligand.

Preferred methods include embodiments wherein said mobilizing agent is Mozabil™.

Preferred methods include embodiments wherein said regenerative activity is angiogenesis.

Preferred methods include embodiments wherein said angiogenesis is production of new blood vessels, which restore circulation to an area of ischemia.

Preferred methods include embodiments wherein said angiogenesis is associated with activation of matrix metalloproteases.

Preferred methods include embodiments wherein said angiogenesis is associated with activation of endothelial cell migration.

Preferred methods include embodiments wherein said angiogenesis is associated with formation of tubules comprising of endothelial cells and pericytes.

Preferred methods include embodiments, wherein said angiogenesis is associated with activation of macrophages possessing the M2 phenotype.

Preferred methods include embodiments wherein said M2 macrophages possess a suppressed expression of inducible nitric oxide synthase as compared to a naïve macrophage.

Preferred methods include embodiments wherein said M2 macrophages possess an enhanced expression of arginase as compared to a naïve macrophage.

Preferred methods include embodiments wherein said M2 macrophages possess an enhanced expression of indolamine 2,3-deoxygenase as compared to a naïve macrophage.

Preferred methods include embodiments wherein said angiogenesis is associated with enhanced production of VEGF.

Preferred methods include embodiments wherein said angiogenesis is associated with enhanced production of SDF-1.

Preferred methods include embodiments wherein said angiogenesis is associated with enhanced production of FGF-1.

Preferred methods include embodiments wherein said angiogenesis is associated with enhanced production of FGF-2.

Preferred methods include embodiments wherein said angiogenesis is associated with enhanced production of FGF-5.

Preferred methods include embodiments wherein said angiogenesis is associated with enhanced production of HGF-1.

Preferred methods include embodiments wherein said angiogenesis is enhanced in response to induction of HIF-1 alpha translocation.

Preferred methods include embodiments wherein said angiogenesis is utilized to accelerate healing.

Preferred methods include embodiments wherein said angiogenesis is further enhanced by culture of cells in the presence of an inhibitor of ROCK.

Preferred methods include embodiments wherein said angiogenesis is further enhanced by culture of cells in the presence of an acidic environment.

Preferred methods include embodiments wherein said angiogenesis is further enhanced by culture of cells in the presence of a hypoxic environment.

Preferred methods include embodiments wherein said angiogenesis is further enhanced by culture of cells in the presence of 5-azacytidine.

Preferred methods include embodiments wherein said angiogenesis is further enhanced by culture of cells in the presence of trichostatin-A.

Preferred methods include embodiments wherein said angiogenesis is further enhanced by culture of cells in the presence of PDGF-BB.

Preferred methods include embodiments wherein said angiogenesis is further enhanced by culture of cells in the presence of PDGF-AA.

Preferred methods include embodiments wherein said angiogenesis is further enhanced by culture of cells in the presence of EGF.

Preferred methods include embodiments wherein said angiogenesis is further enhanced by culture of cells in the presence of IGF.

Preferred methods include embodiments wherein said angiogenesis is further enhanced by culture of cells in the presence of TGF-beta.

Preferred methods include embodiments wherein said angiogenesis is further enhanced by culture of cells in the presence of monocyte conditioned media.

Preferred methods include embodiments wherein said angiogenesis is further enhanced by culture of cells in the presence of dopamine.

Preferred methods include embodiments wherein said regenerative activity is suppression of inflammatory activity.

Preferred methods include embodiments wherein suppression of inflammatory activity is inhibition of NF-kappa B translocation.

Preferred methods include embodiments wherein suppression of inflammatory activity is inhibition of dendritic cell maturation.

Preferred methods include embodiments wherein said dendritic cell maturation is ability to stimulate activation of a naïve T cell.

Preferred methods include embodiments wherein said dendritic cell maturation is ability to stimulate activation of cytokine production in a naïve T cell.

Preferred methods include embodiments wherein said dendritic cell maturation is ability to stimulate cytotoxic activity from a naïve T cell.

Preferred methods include embodiments wherein said dendritic cell maturation is ability to stimulate immunological memory.

Preferred methods include embodiments wherein suppression of inflammatory activity is inhibition of TNF-alpha production.

Preferred methods include embodiments wherein suppression of inflammatory activity is inhibition of interleukin-1 production.

Preferred methods include embodiments wherein suppression of inflammatory activity is inhibition of interleukin-6 production.

Preferred methods include embodiments wherein suppression of inflammatory activity is inhibition of interleukin-8 production.

Preferred methods include embodiments wherein suppression of inflammatory activity is inhibition of interleukin-17 production.

Preferred methods include embodiments wherein said regenerative activity is stimulation of neurogenesis.

Preferred methods include embodiments wherein said regenerative activity is prevention of apoptotic death of cells surrounding administered mesenchymal stem cells.

Preferred methods include embodiments wherein said regenerative activity is stimulation of endogenous progenitor cells.

Preferred methods include embodiments wherein said mTOR inhibitor is rapamycin.

Preferred methods include embodiments wherein said mTOR inhibitor is everolimus.

Preferred methods include embodiments wherein said mTOR inhibitor is ridaforolimus.

### BRIEF DESCRIPTION OF THE DRAWINGS

FIG. 1 is a line graph showing Hepatocyte Growth Factor (HGF-1) expression based on different concentrations of rapamycin

### DETAILED DESCRIPTION OF THE INVENTION

The invention teaches that administration of mTOR inhibitors, such as rapamycin, to mesenchymal stem cells, allows for the generation of enhanced therapeutic activity.

The invention demonstrates that administration of rapamycin, one type of mTOR inhibitor, leads to upregulation of hepatocyte growth factor (HGF-1) production. As used herein, “mTOR inhibitor” refers to any agent that inhibits signaling of mTOR. An mTOR inhibitor is preferably water-soluble. This is because, unless an mTOR inhibitor is water-soluble, it may be necessary to use a solvent that is not highly biocompatible. Water-solubility can be classified based on the definition of solubility in the pharmacopoeia. In other words, the amount of solvent required to dissolve 1 g or 1 mL of solute is defined as extremely readily dissolvable: less than 1 mL; readily dissolvable: 1 mL or greater and less than 10 mL; somewhat readily dissolvable: 10 mL or greater and less than 30 mL; somewhat difficult to dissolve: 30 mL or greater and less than 100 mL; difficult to dissolve: 100 mL or greater and less than 1000 mL; very difficult to dissolve: 1000 mL or greater and less than 10000 mL; and hardly dissolvable: 10000 mL or greater.

The invention teaches that regenerative activities of MSC, which are desired to be upregulated by the teachings of the current invention, are production of growth factors, stimulation of angiogenesis, stimulation of neurogenesis, suppression of inflammation, stimulation of endogenous regenerative cells, enhancement of endothelial reactivity, and prevention of apoptosis. In one embodiment, wherein production of growth factors by MSC are assessed before and after treatment with mTOR inhibitors is disclosed in the current invention. Said growth factors of interest for the purpose of the invention include numerous proteins and peptides which are known to be therapeutic including but not necessarily limited to BLC, Eotaxin-1, Eotaxin-2, G-CSF, GM-CSF, I-309, ICAM-1, IL-1 ra, IL-2, IL-4, IL-5, IL-6 sR, IL-7, IL-10, IL-13, IL-16, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-1ACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TACI, TFPI, TSP-1, TRAIL R2, TRANCE, Troponin I, uPA, VE-Cadherin, WISP-1, and RANK.

In some specific embodiments, specific growth factors are of interest. We will discuss some growth factors for the purpose of assisting one of skill in the art in the practice of the invention. In one embodiment, the enhancement of hepatocyte growth factor (HGF) is desired. HGF has been demonstrated to support generation of immune regulatory cells termed T regulatory cells, which are capable of suppressing various types of inflammation and autoimmunity [1-6]. The potency of HGF acting as an immune modulator is observed in a study in which neutralization of this cytokine resulted in abrogation of several of the therapeutic effects of mesenchymal stem cells (MSC). In the study, MSCs were added to the upper chambers of cell culture inserts, and CD4+ T cells were plated in the lower chambers, followed by treatment with LPS or an anti-HGF antibody. Th17 and Treg cell frequencies were analyzed by flow cytometry, and the expression of Th17 cell- and Treg cell-related cytokines in the CD4+ T cells or culture medium was measured by quantitative PCR (qPCR) and enzyme-linked immunosorbent assay (ELISA), respectively. Neutrophil functions were determined by flow cytometry after a co-culture with Th17/Treg cells. It was found that co-culture with MSC resulted in an increase in Treg and decrease in Th17, which has significantly been inhibited by the anti-HGF antibody. MSCs significantly inhibited the CD4+ T cell expression of IL-17 and IL-6 but increased the expression of IL-10, which was inhibited by the anti-HGF antibody. Additionally, CD4+ T cells co-cultured with MSCs significantly inhibited neutrophil phagocytic and oxidative burst activities and these MSC-induced effects were inhibited by the anti-HGF antibody [7]. Other therapeutic properties of HGF include: stimulation of liver regeneration [8-10], stimulation of renal tubular epithelial cell proliferation [11, 12], enhancement of recovery of renal function after injury [13-24], stimulation of keratinocyte growth [25], stimulation of angiogenesis [26-46], inhibition of cancer cell proliferation [47-54], stimulation of hematopoiesis [55-63], enhancement of B cell activity [64], stimulation of bronchial epithelial cell growth [65, 66], stimulation of type 2 alveolar epithelial cells [67-72], inhibitory of epithelial cell apoptosis [70, 73, 74], stimulation of lung healing [75-80], reduction of pulmonary fibrosis [81-84], enhancement of pancreatic regeneration [85-90], promotion of survival of neurons [91-95], promotion of axonal growth [96], reduction of stroke size and acceleration of recovery [97-99], suppression of neuronal death [100-102], increases brain hypoperfusion [103], inhibits progression of neurodegenerative diseases [104-106], generates more oligodendrocytes [107], attenuates ischemia associated learning dysfunction [108, 109], enhances synaptic plasticity [110], stimulation of neuronal migration [111], stimulation of synaptic localization of receptors [112], activation of muscle satellite cells [113-116], accelerates reconstitution of intestinal epithelial cells [117], accelerate post cardiac infarct recovery [118-134], suppresses cardiomyopathy [135-138], inhibits autoimmune myocarditis [139], reduces endothelial cell injury [140], reduces graft versus host disease [141], improves efficacy of islet transplantation [142-144], restoration of hearing impairment [145, 146], suppression of inflammatory bowel disease [147-150], protects against blindness [151-153], stimulates production of interleukin 1 receptor antagonist [154], accelerates fracture repair [155], suppresses dendritic cell activation/generates tolerogenic dendritic cells [1, 156], promotes recovery after spinal cord injury [157], suppresses autoimmune arthritis [158], and vocal fold scarring [159].

An mTOR (mammalian target of rapamycin) is a serine/threonine kinase identified as a target molecule of rapamycin and is considered to play a central role in the adjustment of cell division, survival and the like. An mTOR is also known as SKS; FRAP; FRAP1; FRAP2; RAFT1; RAPT1, and 2475 is given as a Gene ID of NCBI. Based on such information, those skilled in the art can design and manufacture various mTOR inhibitors.

For the purpose of treatment of MSC, the mTOR inhibitors that can be used in the present invention are not particularly limited, as long as they are compounds having mTOR inhibiting activity. Examples thereof include, rapamycin, temsirolimus, everolimus, PI-103, CC-223, INK128, AZD8055, KU 0063794, Voxtalisib (XL765, SAR245409), Ridaforolimus (Deforolimus, MK-8669), NVP-BEZ235, CZ415, Torkinib (PP242), Torin 1, Omipalisib (GSK2126458, GSK458), OSI-027, PF-04691502, Apitolisib (GDC-0980, RG7422), WYE-354, Vistusertib (AZD2014), Torin 2, Tacrolimus (FK506), GSK1059615, Gedatolisib (PF-05212384, PKI-587), WYE-125132 (WYE-132), BGT226 (NVP-BGT226), Palomid 529 (P529), PP121, WYE-687, CH5132799, WAY-600, ETP-46464, GDC-0349, XL388, Zotarolimus (ABT-578), and Chrysophanic Acid. Preferred mTOR inhibitors include, but are not limited to, rapamycin, temsirolimus and everolimus. Although not wishing to be bound by any theory, this is because these pharmaceutical products are approved by FDA, PMDA, and the like and problems in the aspects of safety and toxicity are minimized. An ever more preferable mTOR inhibitor is rapamycin. Another preferable mTOR inhibitor is temsirolimus. Another preferable mTOR inhibitor is, but is not limited to, everolimus.

Other examples of mTOR inhibitors that can be used in the present invention include neutralizing antibodies against mTORs, compounds inhibiting the activity of mTORs, compounds inhibiting the transcription of a gene encoding an mTOR (e.g., antisense nucleic acids, siRNAs, and ribozymes), peptides, and various compounds. Antisense nucleic acids used in the present invention may inhibit the expression and/or function of a gene (nucleic acids) encoding a member of a signaling pathway of an mTOR or the like by any of the above-described actions. As one embodiment, designing an antisense sequence complementary to an untranslated region near the 5′ end of mRNA of a gene encoding the aforementioned mTOR is considered effective for inhibiting translation of a gene. Further, a sequence that is complementary to an untranslated region of 3′ or a coding region can also be used. In this manner, antisense nucleic acids utilized in the present invention include not only a translation region of a gene encoding the aforementioned mTOR or the like, but also nucleic acids comprising an antisense sequence of a sequence of an untranslated region. An antisense nucleic acid to be used is linked to the downstream of a suitable promoter, and preferably a sequence comprising a transcription termination signal is linked to the 3′ side. A nucleic acid prepared in this manner can be transformed into a desired animal (cell) by using a known method. A sequence of an antisense nucleic acid is preferably a sequence that is complementary to a gene encoding an mTOR of the animal (cell) to be transformed or a portion thereof. However, such a sequence does not need to be fully complementary, as long as gene expression can be effectively suppressed. A transcribed RNA preferably has complementarity that is 90% or greater, and most preferably 95% or greater, with respect to a transcript of a target gene. In order to effectively inhibit the expression of a target gene using an antisense nucleic acid, it is preferable that the length of the antisense nucleic acid is at least 12 bases and less than 25 bases. However, the antisense nucleic acid of the present invention is not necessarily limited to this length. For example, the length may be 11 bases or less, 100 bases or more, or 500 bases or more. An antisense nucleic acid may be composed of only DNA, but may comprise a nucleic acid other than DNAs, such as a locked nucleic acid (LNA). As one embodiment, an antisense nucleic acid used in the present invention may be an LNA containing antisense nucleic acid comprising LNA at the 5′ end or LNA at the 3′ end

Expression of mTOR can also be inhibited by utilizing a ribozyme or DNA encoding a ribozyme. A ribozyme refers to an RNA molecule having catalytic activity. While there are ribozymes with various activities, a study focusing on especially ribozymes as an enzyme for cleaving an RNA made it possible to design a ribozyme that site-specifically cleaves an RNA. There are ribozymes with a size of 400 nucleotides or more as in group I intron ribozymes and M1 RNA contained in RNase P, but there are also those with an active domain of about 40 nucleotides called hammerhead or hair-pin ribozymes.

Expression of an endogenous gene of an mTOR can also be suppressed by RNA interference (hereinafter, abbreviated as “RNAi”) using a double-stranded RNA having a sequence that is identical or similar to a target gene sequence. RNAi is a methodology that is currently drawing attention. The RNAi methodology can suppress the expression of a gene having a sequence that is homologous to a double-stranded RNA (dsRNA) when the dsRNA is incorporated directly into a cell. In mammalian cells, short stranded dsRNA (siRNA) can be used to induce RNAi. RNAi has many advantages over knockout mice, such as a stable effect, facilitated experiment, and low cost. SiRNA is discussed in detail in other parts of the specification.

As used herein “siRNA” is an RNA molecule having a double-stranded RNA portion consisting of 15 to 40 bases, where siRNA has a function of cleaving mRNA of a target gene with a sequence complementary to an antisense strand of the siRNA to suppress the expression of the target gene. Specifically, the siRNA in the present invention is an RNA comprising a double-stranded RNA portion consisting of a sense RNA strand consisting of a sequence homologous to consecutive RNA sequences in mRNA of mTOR and an antisense RNA strand consisting of a sequence complementary to the sense RNA sequence. Design and manufacture of such siRNA and mutant siRNA discussed below are within the technical competence of those skilled in the art. Any consecutive RNA regions of mRNA which is a transcript of a sequence of mTOR can be appropriately selected to make double-stranded RNA corresponding to this region, which is within the ordinary procedure performed by those skilled in the art. Further, those skilled in the art can appropriately select a siRNA sequence having a stronger RNAi effect from mRNA sequences, which are transcripts of the sequence, by a known method. Further, if one of the strands is revealed, those skilled in the art can readily find the base sequence of the other stand (complementary strand). SiRNA can be appropriately made by using a commercially available nucleic acid synthesizer. A common synthesis service can also be utilized for desired RNA synthesis.

In terms of bases, the length of a double-stranded RNA portion is 15 to 40 bases, preferably 15 to 30 bases, more preferably 15 to 25 bases, still more preferably 18 to 23 bases, and most preferably 19 to 21 bases. It is understood that the upper limits and the lower limits thereof are not limited to such specific limits, and may be of any combination of the mentioned limits. The end structure of a sense strand or antisense strand of siRNA is not particularly limited, and can be appropriately selected in accordance with the objective. For example, such an end structure may have a blunt end or a sticky end (overhang). A type where the 3′ end protrudes out is preferred. SiRNA having an overhang consisting of several bases, preferably 1 to 3 bases, and more preferably 2 bases at the 3′ end of a sense RNA strand and antisense RNA strand is preferable for having a large effect of suppressing expression of a target gene in many cases. The type of bases of an overhang is not particularly limited, which may be either a base constituting a RNA or a base constituting a DNA. An example of a preferred overhang sequence includes dTdT at the 3′ end (2 bp of deoxy T) and the like. Examples of preferable siRNA include, but are not limited to, all siRNAs with dTdT (2 bp of deoxy T) at the 3′ end of the sense or antisense strands of the siRNA.

Furthermore, it is also possible to use siRNA in which one to several nucleotides are deleted, substituted, inserted and/or added at one or both of the sense strand and antisense strand of the siRNA described above. One to several bases as used herein is not particularly limited, but preferably refers to 1 to 4 bases, more preferably 1 to bases, and most preferably 1 to 2 bases. Specific examples of such mutations include, but are not limited to, mutations resulting in 0 to 3 bases at the 3′-overhang portion, mutations that change the base sequence of the 3′-overhang portion to another base sequence, mutations in which the lengths of the above-described sense RNA strand and antisense RNA strand are different by 1 to 3 bases due to insertion, addition or deletion of bases, mutations substituting a base in the sense strand and/or the antisense with another base, and the like. However, it is necessary that the sense strand and the antisense strand can hybridize in such mutant siRNAs, and these mutant siRNAs have the ability to suppress gene expression that is equivalent to that of siRNAs without any mutations.

siRNA may also be a molecule with a structure in which one end is closed, such as siRNA with a hairpin structure (Short Hairpin RNA; shRNA). A shRNA is an RNA comprising a sense strand RNA with a specific sequence of a target gene, an antisense strand RNA consisting of a sequence complementary to the sense strand sequence, and a linker sequence for connecting the two strands, wherein the sense strand portion hybridizes with the antisense strand portion to form a double-stranded RNA portion.

It is desirable for siRNA to not exhibit the so-called off-target effect in clinical use. An off-target effect refers to an action for suppressing the expression of another gene, besides the target gene, which is partially homologous to the siRNA used. In order to avoid an off-target effect, it is possible to confirm that a candidate siRNA does not have cross reactivity by determining if there are DNA strands which could react with siRNA by using a DNA microarray or the like in advance. Further, it is possible to avoid an off-target effect by confirming whether there is a gene comprising a moiety that is highly homologous to a sequence of a candidate siRNA, other than a target gene, using a known database provided by the NCBI (National Center for Biotechnology Information) or the like.

### EXAMPLE 1

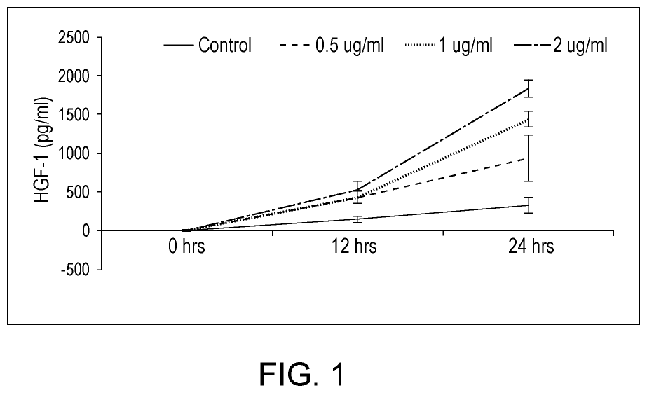

Bone marrow MSCs were purchased from American Type Culture Collection (hereinafter, “ATCC”) and grown in DMEM media with 10% fetal calf serum. Cells were allowed to expand to 100% confluence. The media was subsequently washed with phosphate buffered saline (PBS). Cells were plated on 96 well plates and cultured in the presence of the indicated concentrations of rapamycin for the indicated time points. Hepatocyte Growth Factor (HGF-1) expression was assessed using ELISA (R&D Systems). A significant increase in production of HGF-1 was observed in response to rapamycin treatment as shown in FIG. 1

### REFERENCES

1. Rutella S, Bonanno G, Procoli A, Mariotti A, de Ritis D G, Curti A, Danese S, Pessina G, Pandolfi S, Natoni F et al: Hepatocyte growth factor favors monocyte differentiation into regulatory interleukin (IL)-10++IL-12low/neg accessory cells with dendritic-cell features. Blood 2006, 108(1):218-227.
2. Boumaza I, Srinivasan S, Witt W T, Feghali-Bostwick C, Dai Y, Garcia-Ocana A, Feili-Hariri M: Autologous bone marrow-derived rat mesenchymal stem cells promote PDX-1 and insulin expression in the islets, alter T cell cytokine pattern and preserve regulatory T cells in the periphery and induce sustained normoglycemia. J Autoimmun 2009, 32(1):33-42.
3. Benkhoucha M, Santiago-Raber M L, Schneiter G, Chofflon M, Funakoshi H, Nakamura T, Lalive P H: Hepatocyte growth factor inhibits CNS autoimmunity by inducing tolerogenic dendritic cells and CD25+Foxp3+ regulatory T cells. Proc Natl Acad Sci U S A 2010, 107(14):6424-6429.
4. Demircan P C, Sariboyaci A E, Unal Z S, Gacar G, Subasi C, Karaoz E: Immunoregulatory effects of human dental pulp-derived stem cells on T cells: comparison of transwell co-culture and mixed lymphocyte reaction systems. Cytotherapy 2011, 13(10):1205-1220.
5. Oku M, Okumi M, Shimizu A, Sahara H, Setoyama K, Nishimura H, Sada M, Scalea J, Ido A, Sachs DH et al: Hepatocyte growth factor sustains T regulatory cells and prolongs the survival of kidney allografts in major histocompatibility complex-inbred CLAWN-miniature swine. Transplantation 2012, 93(2):148-155.
6. Gregorini M, Bosio F, Rocca C, Corradetti V, Valsania T, Pattonieri E F, Esposito P, Bedino G, Collesi C, Libetta C et al: Mesenchymal stromal cells reset the scatter factor system and cytokine network in experimental kidney transplantation. BMC Immunol 2014, 15:44.
7. Chen Q H, Wu F, Liu L, Chen H B, Zheng R Q, Wang H L, Yu L N: Mesenchymal stem cells regulate the Th17/Treg cell balance partly through hepatocyte growth factor in vitro. Stem Cell Res Ther 2020, 11(1):91.
8. Kinoshita T, Hirao S, Matsumoto K, Nakamura T: Possible endocrine control by hepatocyte growth factor of liver regeneration after partial hepatectomy. Biochem Biophys Res Commun 1991, 177(1):330-335.
9. Zarnegar R, DeFrances M C, Kost D P, Lindroos P, Michalopoulos G K: Expression of hepatocyte growth factor mRNA in regenerating rat liver after partial hepatectomy. Biochem Biophys Res Commun 1991, 177(1):559-565.
10. Xue F, Takahara T, Yata Y, Minemura M, Morioka C Y, Takahara S, Yamato E, Dono K, Watanabe A: Attenuated acute liver injury in mice by naked hepatocyte growth factor gene transfer into skeletal muscle with electroporation. Gut 2002, 50(4):558-562.
11. Igawa T, Kanda S, Kanetake H, Saitoh Y, Ichihara A, Tomita Y, Nakamura T: Hepatocyte growth factor is a potent mitogen for cultured rabbit renal tubular epithelial cells. Biochem Biophys Res Commun 1991, 174(2):831-838.
12. Harris R C, Burns K D, Alattar M, Homma T, Nakamura T: Hepatocyte growth factor stimulates phosphoinositide hydrolysis and mitogenesis in cultured renal epithelial cells. Life Sci 1993, 52(13):1091-1100.
13. Miller S B, Martin D R, Kissane J, Hammerman M R: Hepatocyte growth factor accelerates recovery from acute ischemic renal injury in rats. Am J Physiol 1994, 266(1 Pt 2):F129-134.
14. Kawaida K, Matsumoto K, Shimazu H, Nakamura T: Hepatocyte growth factor prevents acute renal failure and accelerates renal regeneration in mice. Proc Nail Acad Sci U S A 1994, 91(10):4357-4361.
15. Yang J, Dai C, Liu Y: Systemic administration of naked plasmid encoding hepatocyte growth factor ameliorates chronic renal fibrosis in mice. Gene Ther 2001, 8(19):1470-1479.
16. Dai C, Yang J, Liu Y: Single injection of naked plasmid encoding hepatocyte growth factor prevents cell death and ameliorates acute renal failure in mice. J Am Soc Nephrol 2002, 13(2):411-422.
17. Yamasaki N, Nagano T, Mori-Kudo I, Tsuchida A, Kawamura T, Seki H, Taiji M, Noguchi H: Hepatocyte growth factor protects functional and histological disorders of HgCl(2)-induced acute renal failure mice. Nephron 2002, 90(2):195-205.
18. Nagano T, Mori-Kudo I, Tsuchida A, Kawamura T, Taiji M, Noguchi H: Ameliorative effect of hepatocyte growth factor on glycerol-induced acute renal failure with acute tubular necrosis. Nephron 2002, 91(4):730-738.
19. Gao X, Mae H, Ayabe N, Takai T, Oshima K, Hattori M, Ueki T, Fujimoto J, Tanizawa T: Hepatocyte growth factor gene therapy retards the progression of chronic obstructive nephropathy. Kidney Int 2002, 62(4):1238-1248.
20. Yang J, Dai C, Liu Y: Hepatocyte growth factor gene therapy and angiotensin II blockade synergistically attenuate renal interstitial fibrosis in mice. J Am Soc Nephrol 2002, 13(10):2464-2477.
21. Mori T, Shimizu A, Masuda Y, Fukuda Y, Yamanaka N: Hepatocyte growth factor-stimulating endothelial cell growth and accelerating glomerular capillary repair in experimental progressive glomerulonephritis. Nephron Exp Nephrol 2003, 94(2):e44-54.
22. Okada H, Watanabe Y, Inoue T, Kobayashi T, Kanno Y, Shiota G, Nakamura T, Sugaya T, Fukamizu A, Suzuki H: Transgene-derived hepatocyte growth factor attenuates reactive renal fibrosis in aristolochic acid nephrotoxicity. Nephrol Dial Transplant 2003, 18(12):2515-2523.
23. Dai C, Yang J, Bastacky S, Xia J, Li Y, Liu Y: Intravenous administration of hepatocyte growth factor gene ameliorates diabetic nephropathy in mice. J Am Soc Nephrol 2004, 15(10):2637-2647.
24. Gong R, Rifai A, Tolbert E M, Biswas P, Centracchio J N, Dworkin L D: Hepatocyte growth factor ameliorates renal interstitial inflammation in rat remnant kidney by modulating tubular expression of macrophage chemoattractant protein-1 and RANTES. J Am Soc Nephrol 2004, 15(11):2868-2881.
25. Matsumoto K, Hashimoto K, Yoshikawa K, Nakamura T: Marked stimulation of growth and motility of human keratinocytes by hepatocyte growth factor. Exp Cell Res 1991, 196(1):114-120.
26. Morimoto A, Okamura K, Hamanaka R, Sato Y, Shima N, Higashio K, Kuwano M: Hepatocyte growth factor modulates migration and proliferation of human microvascular endothelial cells in culture. Biochem Biophys Res Commun 1991, 179(2):1042-1049.
27. Bussolino F, Di Renzo M F, Ziche M, Bocchietto E, Olivero M, Naldini L, Gaudino G, Tamagnone L, Coffer A, Comoglio P M: Hepatocyte growth factor is a potent angiogenic factor which stimulates endothelial cell motility and growth. J Cell Biol 1992, 119(3):629-641.
28. Rosen E M, Grant D S, Kleinman H K, Goldberg I D, Bhargava M M, Nickoloff B J, Kinsella J L, Polverini P: Scatter factor (hepatocyte growth factor) is a potent angiogenesis factor in vivo. Symp Soc Exp Biol 1993, 47:227-234.
29. Rosen E M, Lamszus K, Laterra J, Polverini P J, Rubin J S, Goldberg I D: HGF/SF in angiogenesis. Ciba Found Symp 1997, 212:215-226; discussion 227-219.
30. Camussi G, Montrucchio G, Lupia E, Soldi R, Comoglio P M, Bussolino F: Angiogenesis induced in vivo by hepatocyte growth factor is mediated by platelet-activating factor synthesis from macrophages. J Immunol 1997, 158(3):1302-1309.
31. Van Belle E, Witzenbichler B, Chen D, Silver M, Chang L, Schwall R, Isner J M: Potentiated angiogenic effect of scatter factor/hepatocyte growth factor via induction of vascular endothelial growth factor: the case for paracrine amplification of angiogenesis. Circulation 1998, 97(4):381-390.
32. Okada M, Matsumori A, Ono K, Miyamoto T, Takahashi M, Sasayama S: Hepatocyte growth factor is a major mediator in heparin-induced angiogenesis. Biochem Biophys Res Commun 1999, 255(1):80-87.
33. Morishita R, Nakamura S, Hayashi S, Taniyama Y, Moriguchi A, Nagano T, Taiji M, Noguchi H, Takeshita S, Matsumoto K et al: Therapeutic angiogenesis induced by human recombinant hepatocyte growth factor in rabbit hind limb ischemia model as cytokine supplement therapy. Hypertension 1999, 33(6):1379-1384.
34. Wang H, Keiser J A: Hepatocyte growth factor enhances MMP activity in human endothelial cells. Biochem Biophys Res Commun 2000, 272(3):900-905.
35. Xin X, Yang S, Ingle G, Zlot C, Rangell L, Kowalski J, Schwall R, Ferrara N, Gerritsen M E: Hepatocyte growth factor enhances vascular endothelial growth factor-induced angiogenesis in vitro and in vivo. Am J Pathol 2001, 158(3):1111-1120.
36. Taniyama Y, Morishita R, Aoki M, Nakagami H, Yamamoto K, Yamazaki K, Matsumoto K, Nakamura T, Kaneda Y, Ogihara T: Therapeutic angiogenesis induced by human hepatocyte growth factor gene in rat and rabbit hindlimb ischemia models: preclinical study for treatment of peripheral arterial disease. Gene Ther 2001, 8(3):181-189.
37. Aoki M, Morishita R, Taniyama Y, Kaneda Y, Ogihara T: Therapeutic angiogenesis induced by hepatocyte growth factor: potential gene therapy for ischemic diseases. J Atheroscler Thromb 2000, 7(2):71-76.
38. Taniyama Y, Morishita R, Hiraoka K, Aoki M, Nakagami H, Yamasaki K, Matsumoto K, Nakamura T, Kaneda Y, Ogihara T: Therapeutic angiogenesis induced by human hepatocyte growth factor gene in rat diabetic hind limb ischemia model: molecular mechanisms of delayed angiogenesis in diabetes. Circulation 2001, 104(19):2344-2350.
39. Morishita R, Sakaki M, Yamamoto K, Iguchi S, Aoki M, Yamasaki K, Matsumoto K, Nakamura T, Lawn R, Ogihara T et al: Impairment of collateral formation in lipoprotein(a) transgenic mice: therapeutic angiogenesis induced by human hepatocyte growth factor gene. Circulation 2002, 105(12):1491-1496.
40. Nayeri F, Stromberg T, Larsson M, Brudin L, Soderstrom C, Forsberg P: Hepatocyte growth factor may accelerate healing in chronic leg ulcers: a pilot study. J Dermatolog Treat 2002, 13(2):81-86.
41. Sengupta S, Gherardi E, Sellers L A, Wood J M, Sasisekharan R, Fan T P: Hepatocyte growth factor/scatter factor can induce angiogenesis independently of vascular endothelial growth factor. Arterioscler Thromb Vasc Biol 2003, 23(1):69-75.
42. Ding S, Merkulova-Rainon T, Han Z C, Tobelem G: HGF receptor up-regulation contributes to the angiogenic phenotype of human endothelial cells and promotes angiogenesis in vitro. Blood 2003, 101(12):4816-4822.
43. Tomita N, Morishita R, Taniyama Y, Koike H, Aoki M, Shimizu H, Matsumoto K, Nakamura T, Kaneda Y, Ogihara T: Angiogenic property of hepatocyte growth factor is dependent on upregulation of essential transcription factor for angiogenesis, ets-1. Circulation 2003, 107(10):1411-1417.
44. Zhang Y W, Su Y, Volpert O V, Vande Woude G F: Hepatocyte growth factor/scatter factor mediates angiogenesis through positive VEGF and negative thrombospondin 1 regulation. Proc Natl Acad Sci U S A 2003, 100(22):12718-12723.
45. Yamaguchi T, Sawa Y, Miyamoto Y, Takahashi T, Jau CC, Ahmet I, Nakamura T, Matsuda H: Therapeutic angiogenesis induced by injecting hepatocyte growth factor in ischemic canine hearts. Surg Today 2005, 35(10):855-860.
46. Wang W, Yang Z J, Ma D C, Wang L S, Xu S L, Zhang Y R, Cao K J, Zhang F M, Ma W Z: Induction of collateral artery growth and improvement of post-infarct heart function by hepatocyte growth factor gene transfer. Acta Pharmacol Sin 2006, 27(5):555-560.
47. Tajima H, Matsumoto K, Nakamura T: Hepatocyte growth factor has potent anti-proliferative activity in various tumor cell lines. FEBS Lett 1991, 291(2):229-232.
48. Hatano M, Nakata K, Nakao K, Tsutsumi T, Ohtsuru A, Nakamura T, Tamaoki T, Nagataki S: Hepatocyte growth factor down-regulates the alpha-fetoprotein gene expression in PLC/PRF/5 human hepatoma cells. Biochem Biophys Res Commun 1992, 189(1):385-391.
49. Higashio K, Shima N: Tumor cytotoxic activity of HGF-SF. EXS 1993, 65:351-368.
50. Shiota G, Kawasaki H, Nakamura T, Schmidt E V: Inhibitory effect of hepatocyte growth factor on metastasis of hepatocellular carcinoma in transgenic mice. Res Commun Mol Pathol Pharmacol 1996, 91(1):33-39.
51. Yanagawa K, Yamashita T, Yada K, Ohira M, Ishikawa T, Yano Y, Otani S, Sowa M: The antiproliferative effect of HGF on hepatoma cells involves induction of apoptosis with increase in intracellular polyamine concentration levels. Oncol Rep 1998, 5(1):185-190.
52. Arakaki N, Kazi J A, Kazihara T, Ohnishi T, Daikuhara Y: Hepatocyte growth factor/scatter factor activates the apoptosis signaling pathway by increasing caspase-3 activity in sarcoma 180 cells. Biochem Biophys Res Commun 1998, 245(1):211-215.
53. Tsunoda Y, Shibusawa M, Tsunoda A, Gomi A, Yatsuzuka M, Okamatsu T: Antitumor effect of hepatocyte growth factor on hepatoblastoma. Anticancer Res 1998, 18(6A):4339-4342.
54. Yuge K, Takahashi T, Nagano S, Terazaki Y, Murofushi Y, Ushikoshi H, Kawai T, Khai N C, Nakamura T, Fujiwara H et al: Adenoviral gene transduction of hepatocyte growth factor elicits inhibitory effects for hepatoma. Int J Oncol 2005, 27(1):77-85.
55. Kmiecik T E, Keller J R, Rosen E, Vande Woude G F: Hepatocyte growth factor is a synergistic factor for the growth of hematopoietic progenitor cells. Blood 1992, 80(10):2454-2457.
56. Mizuno K, Higuchi O, Ihle J N, Nakamura T: Hepatocyte growth factor stimulates growth of hematopoietic progenitor cells. Biochem Biophys Res Commun 1993, 194(1):178-186.
57. Galimi F, Bagnara G P, Bonsi L, Cottone E, Follenzi A, Simeone A, Comoglio P M: Hepatocyte growth factor induces proliferation and differentiation of multipotent and erythroid hemopoietic progenitors. J Cell Biol 1994, 127(6 Pt 1):1743-1754.
58. Nishino T, Hisha H, Nishino N, Adachi M, Ikehara S: Hepatocyte growth factor as a hematopoietic regulator. Blood 1995, 85(11):3093-3100.
59. Goff J P, Shields D S, Petersen B E, Zajac V F, Michalopoulos G K, Greenberger J S: Synergistic effects of hepatocyte growth factor on human cord blood CD34+ progenitor cells are the result of c-met receptor expression. Stem Cells 1996, 14(5):592-602.
60. Takai K, Hara J, Matsumoto K, Hosoi G, Osugi Y, Tawa A, Okada S, Nakamura T: Hepatocyte growth factor is constitutively produced by human bone marrow stromal cells and indirectly promotes hematopoiesis. Blood 1997, 89(5):1560-1565.
61. Weimar I S, Miranda N, Muller E J, Hekman A, Kerst J M, de Gast G C, Gerritsen W R: Hepatocyte growth factor/scatter factor (HGF/SF) is produced by human bone marrow stromal cells and promotes proliferation, adhesion and survival of human hematopoietic progenitor cells (CD34+). Exp Hematol 1998, 26(9):885-894.
62. Iguchi T, Sogo S, Hisha H, Taketani S, Adachi Y, Miyazaki R, Ogata H, Masuda S, Sasaki R, Ito M et al: HGF activates signal transduction from EPO receptor on human cord blood CD34+/CD45+ cells. Stem Cells 1999, 17(2):82-91.
63. Sugiura K, Taketani S, Yoshimura T, Nishino T, Nishino N, Fujisawa J, Hisha H, Inaba T, Ikehara S: Effect of hepatocyte growth factor on long term hematopoiesis of human progenitor cells in transgenic-severe combined immunodeficiency mice. Cytokine 2007, 37(3):218-226.
64. Delaney B, Koh W S, Yang K H, Strom S C, Kaminski N E: Hepatocyte growth factor enhances B-cell activity. Life Sci 1993, 53(5):PL89-93.
65. Tsao M S, Zhu H, Giaid A, Viallet J, Nakamura T, Park M: Hepatocyte growth factor/scatter factor is an autocrine factor for human normal bronchial epithelial and lung carcinoma cells. Cell Growth Differ 1993, 4(7):571-579.
66. Okada M, Sugita K, Inukai T, Goi K, Kagami K, Kawasaki K, Nakazawa S: Hepatocyte growth factor protects small airway epithelial cells from apoptosis induced by tumor necrosis factor-alpha or oxidative stress. Pediatr Res 2004, 56(3):336-344.
67. Panos R J, Rubin J S, Csaky K G, Aaronson S A, Mason R J: Keratinocyte growth factor and hepatocyte growth factor/scatter factor are heparin-binding growth factors for alveolar type II cells in fibroblast-conditioned medium. J Clin Invest 1993, 92(2):969-977.
68. Mason R J, Leslie C C, McCormick-Shannon K, Deterding R R, Nakamura T, Rubin J S, Shannon J M: Hepatocyte growth factor is a growth factor for rat alveolar type II cells. Am J Respir Cell Mol Biol 1994, 11(5):561-567.
69. Shiratori M, Michalopoulos G, Shinozuka H, Singh G, Ogasawara H, Katyal S L: Hepatocyte growth factor stimulates DNA synthesis in alveolar epithelial type II cells in vitro. Am J Respir Cell Mol Biol 1995, 12(2):171-180.
70. Longati P, Albero D, Comoglio P M: Hepatocyte growth factor is a pleiotropic factor protecting epithelial cells from apoptosis. Cell Death Differ 1996, 3(1):23-28.
71. Panos R J, Patel R, Bak P M: Intratracheal administration of hepatocyte growth factor/scatter factor stimulates rat alveolar type II cell proliferation in vivo. Am J Respir Cell Mol Biol 1996, 15(5):574-581.
72. Itakura A, Kurauchi O, Morikawa S, Furugori K, Mizutani S, Tomoda Y: Human amniotic fluid motogenic activity for fetal alveolar type II cells by way of hepatocyte growth factor. Obstet Gynecol 1997, 89(5 Pt 1):729-733.
73. Yo Y, Morishita R, Nakamura S, Tomita N, Yamamoto K, Moriguchi A, Matsumoto K, Nakamura T, Higaki J, Ogihara T: Potential role of hepatocyte growth factor in the maintenance of renal structure: anti-apoptotic action of HGF on epithelial cells. Kidney Int 1998, 54(4):1128-1138.
74. Zhou Y J, Wang J H, Zhang J: Hepatocyte growth factor protects against apoptosis induced by advanced glycation end products in endothelial cells. Chin Med Sci J 2006, 21(1):6-10.
75. Yanagita K, Matsumoto K, Sekiguchi K, Ishibashi H, Niho Y, Nakamura T: Hepatocyte growth factor may act as a pulmotrophic factor on lung regeneration after acute lung injury. J Biol Chem 1993, 268(28):21212-21217.
76. Ware L B, Matthay M A: Keratinocyte and hepatocyte growth factors in the lung: roles in lung development, inflammation, and repair. Am J Physiol Lung Cell Mol Physiol 2002, 282(5):L924-940.
77. Sakamaki Y, Matsumoto K, Mizuno S, Miyoshi S, Matsuda H, Nakamura T: Hepatocyte growth factor stimulates proliferation of respiratory epithelial cells during postpneumonectomy compensatory lung growth in mice. Am J Respir Cell Mol Biol 2002, 26(5):525-533.
78. Ono M, Sawa Y, Matsumoto K, Nakamura T, Kaneda Y, Matsuda H: In vivo gene transfection with hepatocyte growth factor via the pulmonary artery induces angiogenesis in the rat lung. Circulation 2002, 106(12 Suppl 1):I264-269.
79. Ishizawa K, Kubo H, Yamada M, Kobayashi S, Suzuki T, Mizuno S, Nakamura T, Sasaki H: Hepatocyte growth factor induces angiogenesis in injured lungs through mobilizing endothelial progenitor cells. Biochem Biophys Res Commun 2004, 324(1):276-280.
80. Makiuchi A, Yamaura K, Mizuno S, Matsumoto K, Nakamura T, Amano J, Ito K: Hepatocyte growth factor prevents pulmonary ischemia-reperfusion injury in mice. J Heart Lung Transplant 2007, 26(9):935-943.
81. Dohi M, Hasegawa T, Yamamoto K, Marshall B C: Hepatocyte growth factor attenuates collagen accumulation in a murine model of pulmonary fibrosis. Am J Respir Crit Care Med 2000, 162(6):2302-2307.
82. Watanabe M, Ebina M, Orson F M, Nakamura A, Kubota K, Koinuma D, Akiyama K, Maemondo M, Okouchi S, Tahara M et al: Hepatocyte growth factor gene transfer to alveolar septa for effective suppression of lung fibrosis. Mol Ther 2005, 12(1):58-67.
83. Asano Y, Iimuro Y, Son G, Hirano T, Fujimoto J: Hepatocyte growth factor promotes remodeling of murine liver fibrosis, accelerating recruitment of bone marrow-derived cells into the liver. Hepatol Res 2007, 37(12):1080-1094.
84. Long X, Xiong S D, Xiong W N, Xu Y J: Effect of intramuscular injection of hepatocyte growth factor plasmid DNA with electroporation on bleomycin-induced lung fibrosis in rats. Chin Med J (Engl) 2007, 120(16):1432-1437.
85. Vila M R, Nakamura T, Real F X: Hepatocyte growth factor is a potent mitogen for normal human pancreas cells in vitro. Lab Invest 1995, 73(3):409-418.
86. Mashima H, Shibata H, Mine T, Kojima I: Formation of insulin-producing cells from pancreatic acinar AR42J cells by hepatocyte growth factor. Endocrinology 1996, 137(9):3969-3976.
87. Jeffers M, Rao M S, Rulong S, Reddy J K, Subbarao V, Hudson E, Vande Woude G F, Resau J H: Hepatocyte growth factor/scatter factor-Met signaling induces proliferation, migration, and morphogenesis of pancreatic oval cells. Cell Growth Differ 1996, 7(12):1805-1813.
88. Dai C, Li Y, Yang J, Liu Y: Hepatocyte growth factor preserves beta cell mass and mitigates hyperglycemia in streptozotocin-induced diabetic mice. J Biol Chem 2003, 278(29):27080-27087.
89. Park M K, Kim D K, Lee H J: Adenoviral mediated hepatocyte growth factor gene attenuates hyperglycemia and beta cell destruction in overt diabetic mice. Exp Mol Med 2003, 35(6):494-500.
90. Izumida Y, Aoki T, Yasuda D, Koizumi T, Suganuma C, Saito K, Murai N, Shimizu Y, Hayashi K, Odaira M et al: Hepatocyte growth factor is constitutively produced by donor-derived bone marrow cells and promotes regeneration of pancreatic beta-cells. Biochem Biophys Res Commun 2005, 333(1):273-282.
91. Wong V, Glass D J, Arriaga R, Yancopoulos G D, Lindsay R M, Conn G: Hepatocyte growth factor promotes motor neuron survival and synergizes with ciliary neurotrophic factor. J Biol Chem 1997, 272(8):5187-5191.
92. Maina F, Hilton M C, Ponzetto C, Davies A M, Klein R: Met receptor signaling is required for sensory nerve development and HGF promotes axonal growth and survival of sensory neurons. Genes Dev 1997, 11(24):3341-3350.
93. Miyazawa T, Matsumoto K, Ohmichi H, Katoh H, Yamashima T, Nakamura T: Protection of hippocampal neurons from ischemia-induced delayed neuronal death by hepatocyte growth factor: a novel neurotrophic factor. J Cereb Blood Flow Metab 1998, 18(4):345-348.
94. Maina F, Hilton M C, Andres R, Wyatt S, Klein R, Davies A M: Multiple roles for hepatocyte growth factor in sympathetic neuron development. Neuron 1998, 20(5):835-846.
95. Ishihara N, Takagi N, Niimura M, Takagi K, Nakano M, Tanonaka K, Funakoshi H, Matsumoto K, Nakamura T, Takeo S: Inhibition of apoptosis-inducing factor translocation is involved in protective effects of hepatocyte growth factor against excitotoxic cell death in cultured hippocampal neurons. J Neurochem 2005, 95(5):1277-1286.
96. Yang X M, Toma J G, Bamji S X, Belliveau D J, Kohn J, Park M, Miller F D: Autocrine hepatocyte growth factor provides a local mechanism for promoting axonal growth. J Neurosci 1998, 18(20):8369-8381.
97. Tsuzuki N, Miyazawa T, Matsumoto K, Nakamura T, Shima K: Hepatocyte growth factor reduces the infarct volume after transient focal cerebral ischemia in rats. Neurol Res 2001, 23(4):417-424.
98. Tsuzuki N, Miyazawa T, Matsumoto K, Nakamura T, Shima K, Chigasaki H: Hepatocyte growth factor reduces infarct volume after transient focal cerebral ischemia in rats. Acta Neurochir Suppl 2000, 76:311-316.
99. Shimamura M, Sato N, Oshima K, Aoki M, Kurinami H, Waguri S, Uchiyama Y, Ogihara T, Kaneda Y, Morishita R: Novel therapeutic strategy to treat brain ischemia: overexpression of hepatocyte growth factor gene reduced ischemic injury without cerebral edema in rat model. Circulation 2004, 109(3):424-431.
100. Hossain M A, Russell J C, Gomez R, Laterra J: Neuroprotection by scatter factor/hepatocyte growth factor and FGF-1 in cerebellar granule neurons is phosphatidylinositol 3-kinase/akt-dependent and MAPK/CREB-independent. J Neurochem 2002, 81(2):365-378.
101. Thompson J, Dolcet X, Hilton M, Tolcos M, Davies A M: HGF promotes survival and growth of maturing sympathetic neurons by PI-3 kinase- and MAP kinase-dependent mechanisms. Mol Cell Neurosci 2004, 27(4):441-452.
102. He F, Wu L X, Shu K X, Liu F Y, Yang L J, Zhou X, Zhang Y, Huang B S, Huang D, Deng X L: HGF protects cultured cortical neurons against hypoxia/reoxygenation induced cell injury via ERK1/2 and PI-3K/Akt pathways. Colloids Surf B Biointerfaces 2008, 61(2):290-297.
103. Yoshimura S, Morishita R, Hayashi K, Kokuzawa J, Aoki M, Matsumoto K, Nakamura T, Ogihara T, Sakai N, Kaneda Y: Gene transfer of hepatocyte growth factor to subarachnoid space in cerebral hypoperfusion model. Hypertension 2002, 39(5):1028-1034.
104. Sun W, Funakoshi H, Nakamura T: Overexpression of HGF retards disease progression and prolongs life span in a transgenic mouse model of ALS. J Neurosci 2002, 22(15):6537-6548.
105. Kadoyama K, Funakoshi H, Ohya W, Nakamura T: Hepatocyte growth factor (HGF) attenuates gliosis and motoneuronal degeneration in the brainstem motor nuclei of a transgenic mouse model of ALS. Neurosci Res 2007, 59(4):446-456.
106. Ishigaki A, Aoki M, Nagai M, Warita H, Kato S, Kato M, Nakamura T, Funakoshi H, Itoyama Y: Intrathecal delivery of hepatocyte growth factor from amyotrophic lateral sclerosis onset suppresses disease progression in rat amyotrophic lateral sclerosis model. J Neuropathol Exp Neurol 2007, 66(11):1037-1044.
107. Yan H, Rivkees S A: Hepatocyte growth factor stimulates the proliferation and migration of oligodendrocyte precursor cells. J Neurosci Res 2002, 69(5):597-606.
108. Date I, Takagi N, Takagi K, Kago T, Matsumoto K, Nakamura T, Takeo S: Hepatocyte growth factor attenuates cerebral ischemia-induced learning dysfunction. Biochem Biophys Res Commun 2004, 319(4):1152-1158.
109. Date I, Takagi N, Takagi K, Kago T, Matsumoto K, Nakamura T, Takeo S: Hepatocyte growth factor improved learning and memory dysfunction of microsphere-embolized rats. J Neurosci Res 2004, 78(3):442-453.
110. Akimoto M, Baba A, Ikeda-Matsuo Y, Yamada M K, Itamura R, Nishiyama N, Ikegaya Y, Matsuki N: Hepatocyte growth factor as an enhancer of nmda currents and synaptic plasticity in the hippocampus. Neuroscience 2004, 128(1):155-162.
111. Cacci E, Salani M, Anastasi S, Perroteau I, Poiana G, Biagioni S, Augusti-Tocco G: Hepatocyte growth factor stimulates cell motility in cultures of the striatal progenitor cells ST14A. J Neurosci Res 2003, 74(5):760-768.
112. Akita H, Takagi N, Ishihara N, Takagi K, Murotomi K, Funakoshi H, Matsumoto K, Nakamura T, Takeo S: Hepatocyte growth factor improves synaptic localization of the NMDA receptor and intracellular signaling after excitotoxic injury in cultured hippocampal neurons. Exp Neurol 2008, 210(1):83-94.
113. Tatsumi R, Anderson J E, Nevoret C J, Halevy O, Allen R E: HGF/SF is present in normal adult skeletal muscle and is capable of activating satellite cells. Dev Biol 1998, 194(1):114-128.
114. Gal-Levi R, Leshem Y, Aoki S, Nakamura T, Halevy O: Hepatocyte growth factor plays a dual role in regulating skeletal muscle satellite cell proliferation and differentiation. Biochim Biophys Acta 1998, 1402(1):39-51.
115. Sheehan S M, Tatsumi R, Temm-Grove C J, Allen R E: HGF is an autocrine growth factor for skeletal muscle satellite cells in vitro. Muscle Nerve 2000, 23(2):239-245.
116. Miller K J, Thaloor D, Matteson S, Pavlath G K: Hepatocyte growth factor affects satellite cell activation and differentiation in regenerating skeletal muscle. Am J Physiol Cell Physiol 2000, 278(1):C174-181.
117. Nishimura S, Takahashi M, Ota S, Hirano M, Hiraishi H: Hepatocyte growth factor accelerates restitution of intestinal epithelial cells. J Gastroenterol 1998, 33(2):172-178.
118. Aoki M, Morishita R, Taniyama Y, Kida I, Moriguchi A, Matsumoto K, Nakamura T, Kaneda Y, Higaki J, Ogihara T: Angiogenesis induced by hepatocyte growth factor in non-infarcted myocardium and infarcted myocardium: up-regulation of essential transcription factor for angiogenesis, ets. Gene Ther 2000, 7(5):417-427.
119. Taniyama Y, Morishita R, Nakagami H, Moriguchi A, Sakonjo H, Shokei K, Matsumoto K, Nakamura T, Higaki J, Ogihara T: Potential contribution of a novel antifibrotic factor, hepatocyte growth factor, to prevention of myocardial fibrosis by angiotensin II blockade in cardiomyopathic hamsters. Circulation 2000, 102(2):246-252.
120. Nakamura T, Mizuno S, Matsumoto K, Sawa Y, Matsuda H, Nakamura T: Myocardial protection from ischemia/reperfusion injury by endogenous and exogenous HGF. J Clin Invest 2000, 106(12):1511-1519.
121. Ueda H, Nakamura T, Matsumoto K, Sawa Y, Matsuda H, Nakamura T: A potential cardioprotective role of hepatocyte growth factor in myocardial infarction in rats. Cardiovasc Res 2001, 51(1):41-50.
122. Kitta K, Day R M, Ikeda T, Suzuki Y J: Hepatocyte growth factor protects cardiac myocytes against oxidative stress-induced apoptosis. Free Radic Biol Med 2001, 31(7):902-910.
123. Taniyama Y, Morishita R, Aoki M, Hiraoka K, Yamasaki K, Hashiya N, Matsumoto K, Nakamura T, Kaneda Y, Ogihara T: Angiogenesis and antifibrotic action by hepatocyte growth factor in cardiomyopathy. Hypertension 2002, 40(1):47-53.
124. Jin H, Yang R, Li W, Ogasawara A K, Schwall R, Eberhard D A, Zheng Z, Kahn D, Paoni N F: Early treatment with hepatocyte growth factor improves cardiac function in experimental heart failure induced by myocardial infarction. J Pharmacol Exp Ther 2003, 304(2):654-660.
125. Ahmet I, Sawa Y, Yamaguchi T, Matsuda H: Gene transfer of hepatocyte growth factor improves angiogenesis and function of chronic ischemic myocardium in canine heart. Ann Thorac Surg 2003, 75(4):1283-1287.
126. Li Y, Takemura G, Kosai K, Yuge K, Nagano S, Esaki M, Goto K, Takahashi T, Hayakawa K, Koda M et al: Postinfarction treatment with an adenoviral vector expressing hepatocyte growth factor relieves chronic left ventricular remodeling and dysfunction in mice. Circulation 2003, 107(19):2499-2506.
127. Jayasankar V, Woo Y J, Bish L T, Pirolli T J, Chatterjee S, Berry M F, Burdick J, Gardner T J, Sweeney H L: Gene transfer of hepatocyte growth factor attenuates postinfarction heart failure. Circulation 2003, 108 Suppl 1:II230-236.
128. Komamura K, Tatsumi R, Miyazaki J, Matsumoto K, Yamato E, Nakamura T, Shimizu Y, Nakatani T, Kitamura S, Tomoike H et al: Treatment of dilated cardiomyopathy with electroporation of hepatocyte growth factor gene into skeletal muscle. Hypertension 2004, 44(3):365-371.
129. Jin H, Wyss J M, Yang R, Schwall R: The therapeutic potential of hepatocyte growth factor for myocardial infarction and heart failure. Curr Pharm Des 2004, 10(20):2525-2533.
130. Kondo I, Ohmori K, Oshita A, Takeuchi H, Fuke S, Shinomiya K, Noma T, Namba T, Kohno M: Treatment of acute myocardial infarction by hepatocyte growth factor gene transfer: the first demonstration of myocardial transfer of a “functional” gene using ultrasonic microbubble destruction. J Am Coll Cardiol 2004, 44(3):644-653.
131. Ryugo M, Sawa Y, Ono M, Fukushima N, Aleshin A N, Mizuno S, Nakamura T, Matsuda H: Myocardial protective effect of human recombinant hepatocyte growth factor for prolonged heart graft preservation in rats. Transplantation 2004, 78(8):1153-1158.
132. Jayasankar V, Woo Y J, Pirolli T J, Bish L T, Berry M F, Burdick J, Gardner T J, Sweeney H L: Induction of angiogenesis and inhibition of apoptosis by hepatocyte growth factor effectively treats postischemic heart failure. J Card Surg 2005, 20(1):93-101.
133. Yang Z, Wang W, Ma D, Zhang Y, Wang L, Zhang Y, Xu S, Chen B, Miao D, Cao K et al: Recruitment of stem cells by hepatocyte growth factor via intracoronary gene transfection in the postinfarction heart failure. Sci China C Life Sci 2007, 50(6):748-752.
134. Chen X H, Minatoguchi S, Kosai K, Yuge K, Takahashi T, Arai M, Wang N, Misao Y, Lu C, Onogi H et al: In vivo hepatocyte growth factor gene transfer reduces myocardial ischemia-reperfusion injury through its multiple actions. J Card Fail 2007, 13(10):874-883.
135. Nakamura T, Matsumoto K, Mizuno S, Sawa Y, Matsuda H, Nakamura T: Hepatocyte growth factor prevents tissue fibrosis, remodeling, and dysfunction in cardiomyopathic hamster hearts. Am J Physiol Heart Circ Physiol 2005, 288(5):H2131-2139.
136. Iwasaki M, Adachi Y, Nishiue T, Minamino K, Suzuki Y, Zhang Y, Nakano K, Koike Y, Wang J, Mukaide H et al: Hepatocyte growth factor delivered by ultrasound-mediated destruction of microbubbles induces proliferation of cardiomyocytes and amelioration of left ventricular contractile function in Doxorubicin-induced cardiomyopathy. Stem Cells 2005, 23(10):1589-1597.
137. Azuma J, Taniyama Y, Takeya Y, lekushi K, Aoki M, Dosaka N, Matsumoto K, Nakamura T, Ogihara T, Morishita R: Angiogenic and antifibrotic actions of hepatocyte growth factor improve cardiac dysfunction in porcine ischemic cardiomyopathy. Gene Ther 2006, 13(16):1206-1213.
138. Esaki M, Takemura G, Kosai K, Takahashi T, Miyata S, Li L, Goto K, Maruyama R, Okada H, Kanamori H et al: Treatment with an adenoviral vector encoding hepatocyte growth factor mitigates established cardiac dysfunction in doxorubicin-induced cardiomyopathy. Am J Physiol Heart Circ Physiol 2008, 294(2):H1048-1057.
139. Futamatsu H, Suzuki J, Mizuno S, Koga N, Adachi S, Kosuge H, Maejima Y, Hirao K, Nakamura T, Isobe M: Hepatocyte growth factor ameliorates the progression of experimental autoimmune myocarditis: a potential role for induction of T helper 2 cytokines. Circ Res 2005, 96(8):823-830.
140. Yasuda S, Noguchi T, Gohda M, Arai T, Tsutsui N, Matsuda T, Nonogi H: Single low-dose administration of human recombinant hepatocyte growth factor attenuates intimal hyperplasia in a balloon-injured rabbit iliac artery model. Circulation 2000, 101(21):2546-2549.
141. Kuroiwa T, Kakishita E, Hamano T, Kataoka Y, Seto Y, Iwata N, Kaneda Y, Matsumoto K, Nakamura T, Ueki T et al: Hepatocyte growth factor ameliorates acute graft-versus-host disease and promotes hematopoietic function. J Clin Invest 2001, 107(11):1365-1373.
142. Garcia-Ocana A, Takane K K, Reddy V T, Lopez-Talavera J C, Vasavada R C, Stewart A F: Adenovirus-mediated hepatocyte growth factor expression in mouse islets improves pancreatic islet transplant performance and reduces beta cell death. J Biol Chem 2003, 278(1):343-351.
143. Beattie G M, Montgomery A M, Lopez A D, Hao E, Perez B, Just M L, Lakey J R, Hart M E, Hayek A: A novel approach to increase human islet cell mass while preserving beta-cell function. Diabetes 2002, 51(12):3435-3439.
144. Lopez-Talavera J C, Garcia-Ocana A, Sipula I, Takane K K, Cozar-Castellano I, Stewart A F: Hepatocyte growth factor gene therapy for pancreatic islets in diabetes: reducing the minimal islet transplant mass required in a glucocorticoid-free rat model of allogeneic portal vein islet transplantation. Endocrinology 2004, 145(2):467-474.
145. Oshima K, Shimamura M, Mizuno S, Tamai K, Doi K, Morishita R, Nakamura T, Kubo T, Kaneda Y: Intrathecal injection of HVJ-E containing HGF gene to cerebrospinal fluid can prevent and ameliorate hearing impairment in rats. FASEB J 2004, 18(1):212-214.
146. Naim R, Shen T, Riedel F, Bran G, Sadick H, Hormann K: Regulation of apoptosis in external auditory canal cholesteatoma by hepatocyte growth factor/scatter factor. ORL J Otorhinolaryngol Relat Spec 2005, 67(1):45-50.
147. Arthur L G, Schwartz M Z, Kuenzler K A, Birbe R: Hepatocyte growth factor treatment ameliorates diarrhea and bowel inflammation in a rat model of inflammatory bowel disease. J Pediatr Surg 2004, 39(2):139-143; discussion 139-143.
148. Oh K, limuro Y, Takeuchi M, Kaneda Y, Iwasaki T, Terada N, Matsumoto T, Nakanishi K, Fujimoto J: Ameliorating effect of hepatocyte growth factor on inflammatory bowel disease in a murine model. Am J Physiol Gastrointest Liver Physiol 2005, 288(4):G729-735.
149. Mukoyama T, Kanbe T, Murai R, Murawaki Y, Shimomura T, Hashiguchi K, Saeki T, Ichiba M, Yoshida Y, Tanabe N et al: Therapeutic effect of adenoviral-mediated hepatocyte growth factor gene administration on TNBS-induced colitis in mice. Biochem Biophys Res Commun 2005, 329(4):1217-1224.
150. Hanawa T, Suzuki K, Kawauchi Y, Takamura M, Yoneyama H, Han G D, Kawachi H, Shimizu F, Asakura H, Miyazaki J et al: Attenuation of mouse acute colitis by naked hepatocyte growth factor gene transfer into the liver. J Gene Med 2006, 8(5):623-635.
151. Machida S, Tanaka M, Ishii T, Ohtaka K, Takahashi T, Tazawa Y: Neuroprotective effect of hepatocyte growth factor against photoreceptor degeneration in rats. Invest Ophthalmol Vis Sci 2004, 45(11):4174-4182.
152. Jin M, Yaung J, Kannan R, He S, Ryan S J, Hinton D R: Hepatocyte growth factor protects RPE cells from apoptosis induced by glutathione depletion. Invest Ophthalmol Vis Sci 2005, 46(11):4311-4319.
153. Ohtaka K, Machida S, Ohzeki T, Tanaka M, Kurosaka D, Masuda T, Ishii T: Protective effect of hepatocyte growth factor against degeneration of the retinal pigment epithelium and photoreceptor in sodium iodate-injected rats. Curr Eye Res 2006, 31(4):347-355.
154. Molnar C, Garcia-Trevijano E R, Ludwiczek O, Talabot D, Kaser A, Mato J M, Fritsche G, Weiss G, Gabay C, Avila M A et al: Anti-inflammatory effects of hepatocyte growth factor: induction of interleukin-1 receptor antagonist. Eur Cytokine Netw 2004, 15(4):303-311.
155. Imai Y, Terai H, Nomura-Furuwatari C, Mizuno S, Matsumoto K, Nakamura T, Takaoka K: Hepatocyte growth factor contributes to fracture repair by upregulating the expression of BMP receptors. J Bone Miner Res 2005, 20(10):1723-1730.
156. Okunishi K, Dohi M, Nakagome K, Tanaka R, Mizuno S, Matsumoto K, Miyazaki J, Nakamura T, Yamamoto K: A novel role of hepatocyte growth factor as an immune regulator through suppressing dendritic cell function. J Immunol 2005, 175(7):4745-4753.
157. Kitamura K, Iwanami A, Nakamura M, Yamane J, Watanabe K, Suzuki Y, Miyazawa D, Shibata S, Funakoshi H, Miyatake S et al: Hepatocyte growth factor promotes endogenous repair and functional recovery after spinal cord injury. J Neurosci Res 2007, 85(11):2332-2342.
158. Okunishi K, Dohi M, Fujio K, Nakagome K, Tabata Y, Okasora T, Seki M, Shibuya M, Imamura M, Harada H et al: Hepatocyte growth factor significantly suppresses collagen-induced arthritis in mice. J Immunol 2007, 179(8):5504-5513.
159. Ohno T, Hirano S, Kanemaru S, Yamashita M, Umeda H, Suehiro A, Tamura Y, Nakamura T, Ito J, Tabata Y: Drug delivery system of hepatocyte growth factor for the treatment of vocal fold scarring in a canine model. Ann Otol Rhinol Laryngol 2007, 116(10):762-769.

1. A method of augmenting regenerative activity of mesenchymal stem cells comprising contacting and mixing said mesenchymal stem cells with one or more inhibitors of mammalian target of rapamycin (mTOR).

2. The method of claim 1, wherein said mesenchymal stem cells express markers selected from a group comprising of: a) CD90; b) CD105 and c) CD74.

3. The method of claim 1, wherein said mesenchymal stem cells lack expression of markers selected from a group comprising of: a) CD14; b) CD45 and c) CD34.

4. The method of claim 1, wherein said mesenchymal stem cells are plastic adherent.

5. The method of claim 1, wherein said mesenchymal stem cells are selected from a group of tissues comprising of: a) bone marrow b) placenta; c) menstrual blood; d) peripheral blood; e) adipose tissue; f) umbilical cord blood; g) Wharton's jelly; and h) fallopian tube.

6. The method of claim 5, wherein said peripheral blood is drawn after subject is treated with one or more agents capable of mobilizing bone marrow derived mesenchymal stem cells.

7. The method of claim 6, wherein said mobilizing agent is G-CSF.

8. The method of claim 6, wherein said mobilizing agent is GM-CSF.

9. The method of claim 6, wherein said mobilizing agent is M-CSF.

10. The method of claim 6, wherein said mobilizing agent is FLT-3 ligand.

11. The method of claim 6, wherein said mobilizing agent is Mozabil™.

12. The method of claim 1, wherein said regenerative activity is angiogenesis.

13. The method of claim 12, wherein said angiogenesis is production of new blood vessels, which restore circulation to an area of ischemia.

14. The method of claim 12, wherein said angiogenesis is associated with activation of matrix metalloproteases.

15. The method of claim 12, wherein said angiogenesis is associated with activation of endothelial cell migration.

16. The method of claim 12, wherein said angiogenesis is associated with formation of tubules comprising of endothelial cells and pericytes.

17. The method of claim 12, wherein said angiogenesis is associated with activation of macrophages possessing the M2 phenotype.

18. The method of claim 1, wherein said mTOR inhibitor is rapamycin.

19. The method of claim 1, wherein said mTOR inhibitor is everolimus.

20. The method of claim 1, wherein said mTOR inhibitor is ridaforolimus
